(19) United States
(12) Patent Application Publication
(10) Pub. No.: US 2009/0304781 A1
(43) Pub. Date: Dec. 10, 2009

(54) MASTER GENE OF ARTERIOGENESIS

(75) Inventors: Wolfgang Schaper, Bad Nauheim (DE); Inka Eitenmüller, Bad Nauheim (DE); Kerstin Troidl, Bad Nauheim (DE); Christian Troidl, Bad Nauheim (DE); Matthias Heil, Ober-Morlen (DE); Thomas Schmitz-Rixen, Frankfurt am Main (DE); Anton J.G. Horrevoets, Nieuwegein (NL)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: MAX-PLANCK-GESELLSCHAFT ZUR FÖRDERUNG DER WISSENSC, Munchen (DE)

(21) Appl. No.: 12/281,467

(22) PCT Filed: Mar. 1, 2007
(86) PCT No.: PCT/EP2007/001774

§ 371 Date: Jul. 27, 2009

(30) Foreign Application Priority Data
Mar. 2, 2006 (EP) 06004221.5
Aug. 25, 2006 (EP) 06017777.1

(51) Int. Cl. A61K 9/127; A61K 38/16; A61K 48/00; A61K 9/00; C12N 5/06; C12Q 1/68; C07K 16/00; A61P 9/10

(52) U.S. Cl. 424/450; 512/12; 424/93.2; 424/422; 435/325; 435/6; 530/387.1

(57) ABSTRACT

The present invention relates to a pharmaceutical composition comprising (a) a polynucleotide comprising or consisting of a nucleotide sequence encoding a polypeptide with the amino acid sequence as set forth in SEQ ID NO: 2; (b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity; (c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or (d) a polypeptide encoded by the polynucleotide of any one of (a) to (c).

This invention relates to a pharmaceutical composition comprising (a) a polynucleotide comprising or consisting of a nucleotide sequence encoding a polypeptide with the amino acid sequence as set forth in SEQ ID NO: 2; (b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity; (c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or (d) a polypeptide encoded by the polynucleotide of any one of (a) to (c).

In this specification, a number of documents including patent applications and manufacturer's manuals is cited. The disclosure of these documents, while not considered relevant for the patentability of this invention, is herewith incorporated by reference in its entirety.

Occlusion of an artery as a complication of underlying atherosclerosis is the major cause of death worldwide. Formerly restricted to the industrialized countries, owing to globalization nowadays emerging countries and those of the third world suffer almost equally from organ manifestations of arterial degeneration and occlusion, affecting hearts, brains, kidneys and the limb circulation. Since only a minority of the affected population dies at the first manifestation of the disease, a long and expensive suffering is the result.

Atherosclerosis can be prevented and, when manifest, treated. However, prevention means that many people may have to abandon a life style that they recognize as a hard won status symbol. The drastic rise of obesity in Western countries, even in those with the “healthy Mediterranean diet”, is a good example of the near futility of recommended lifestyle changes.

Drug therapy can be effective in reducing blood cholesterol levels, but only 25% of the treated population is spared a heart attack.

Heart surgery and catheter-based re-canalizations of arteries are very effective but very expensive and especially stent-based therapies are still prone to re-occlusion.

Angiogenic growth factors had inspired high hopes but clinical trials had been very disappointing. Growth factors are known to stimulate the sprouting of capillaries but capillaries are unable to replace an occluded artery.

The new stem cell based therapies are as yet extremely controversial and recent Scandinavian clinical studies showed them to be inactive in patients with acute myocardial infarction.

During the evolution of the human race, especially when the need arose to walk and run upright, the healing of leg wounds and the replacement of damaged leg arteries became life saving and so a special mechanism for arterial regeneration was developed from which we still benefit: the establishment of a collateral circulation, the formation of “bypass”-vessels in cases of occlusion of the large arteries. This process, which we call now “arteriogenesis”, is operative in the entire arterial system but not as well developed as in the limbs.

Already in the 1970ies it was shown (Schaper et al. (1972), Schaper (1971)) that an arterial occlusion activates the small pre-existent bypass vessels to grow at a very rapid rate: Within one week they were able to furnish normal blood flow to the heart in experimental animals, provided the arterial occlusion proceeded gradually and not abruptly. A limb artery can be acutely occluded but it would take about a week to be able to (slowly) walk again. The process of bypass enlargement is by cell division.

Small bypass vessels can increase their diameter by a factor of 20 times and their tissue mass by a factor of 50 fold. The growth process recruits helper cells from the bone marrow, mainly monocytes but also T-lymphocytes. These cells are necessary to break down the old vascular structure to create the space for the much larger new vessel.

The inventors have previously found that the most important physical force that initiates the growth is the fluid shear stress, i.e., the viscous drag that the flowing blood exerts on the inner lining of the blood vessels, the endothelium (Pipp et al. (2004)). Pipp et al. (2004) used an arterio-venous (AV) shunt in order to create a model system with elevated fluid shear stress. The fluid shear stress is highest immediately after the arterial occlusion because of the blood pressure decrease in the vascular bed downstream of the occlusion, which increases the blood flow velocity and with it the viscous drag. However when the bypass vessels begin to grow, the fluid shear stress falls again because it is inversely related to the 3^{rd} power of the radius. As a consequence the growth of the bypass vessels stops before an ideal adaptation is reached. This had been the limitation of the natural bypass vessel concept. Furthermore, Pipp et al. (2004) conclude their report by stating that the AV shunt approach is likely not applicable for therapeutic purposes because it may increase already existing ischemic states.

In view of the limitations of the methods described in the prior art, the technical problem underlying the present invention was the provision of alternative or improved means and methods for stimulation of arteriogenesis.

Accordingly, this invention relates to a pharmaceutical composition comprising (a) a polynucleotide comprising or consisting of a nucleotide sequence encoding a polypeptide with the amino acid sequence as set forth in SEQ ID NO: 2; (b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity; (c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or (d) a polypeptide encoded by the polynucleotide of any one of (a) to (c).

The term “arteriogenic” as used herein means “causative of arteriogenesis” or “having a stimulatory effect on arteriogenesis”.

The term “arteriogenesis” as used herein relates to the development of arteries from previously existing collateral arterial vessels. The term “collateral arterial vessels” designates arterial vessels which are secondary or subordinate as compared to the principal arterial vessel in a given part of the human or animal body. The terms “secondary” and “subordinate” relate inter alia to the diameter of and the flow rate (amount of blood per unit of time) through the collateral arterial vessels. The process of arteriogenesis leads to a conversion of one or more of said collateral arterial vessels into the main arterial vessel(s) in said part of the body. The conversion comprises an increase in diameter and a concomitant increase in blood flow rate. The increase in diameter may be 2-, 3-, 4-, 5-, 10- or 20-fold or larger. Concomitantly, the tissue mass of the vessel may increase by a factor of 5, 10, 20 or 50 or above. As a consequence, the converted collateral arterial vessel(s) may serve as a replacement for a damaged or non-functional arterial vessel. In other words, arteriogenesis leads to the formation of bypass vessels.

Arteriogenic activity according to the invention may be assayed by methods known in the art (Schaper et al. (1969), Schaper et al. (1971), Pasyk et al. (1982), Schaper et al. (1976)) and as described in the Examples enclosed herewith, including hemodynamic measurements and angiography such as post mortem angiography.

Hemodynamic measurements permit the determination of maximum collateral conductances. The term “maximum collateral conductance” designates the maximum capacity of collateral circulation. The measurements may comprise systemic pressure measurements, for example in the right carotid artery, and/or peripheral pressure measurements, for example in both saphenous arteries. Synchronously, hindlimb blood flow (e.g. in a rabbit model system) may be detected at increasing adenosine concentrations (100 to 600 μg per kg body weight and per min) in both iliac arteries.

Post mortem angiography may be performed as follows (the example relates to the rabbit model system): Hind limbs of euthanized rabbits are perfused with a gelatine-barium-based contract medium. Angiograms of the hind limbs are taken in a Balteau radiography apparatus. Angiographically visible collateral arteries, spanning from the arteriae profundae femoralis and circumflexa femoris lateralis to the arteriae genualis descendens and caudalis femoris are counted.

The term “arterial” as used herein means “pertaining to an artery or to the arteries”. Any type of artery is envisaged. The term “artery” includes aorta (and parts thereof such as ascending aorta, descending aorta, thoracic aorta, abdominal aorta), pulmonary artery, carotid artery (external and internal), arteries of the brain, subclavian artery, axillary artery, brachial artery, radial artery, ulnar artery, hypogastric artery, hepatic artery, iliac artery, femoral artery, popliteal artery, anterior and posterior tibial artery, and arteria dorsalis pedis.

The term “arteriogenesis” is to be distinguished from “angiogenesis”. Angiogenesis relates to the formation of new arterial vessels in a region where previously no or less arterial vessels existed. As such it is different from “arteriogenesis” which designates a conversion of smaller into larger arterial vessels.

The sequence of SEQ ID NO: 2 is the sequence of the human ABRA protein. The symbol ABRA stands for “actin-binding Rho activating protein”. A previously used alias name is STARS. STARS stands for “striated muscle activator of Rho-dependent signalling”. In connection with the present invention the names ABRA and STARS are used interchangeably. The sequence can be found, for example, in the Entrez Protein database maintained by the National Center for Biotechnology Information (NCBI) under the database accession number NP_{—}631905. The sequence of SEQ ID NO: 2 is identical with the sequence provided in database entry NP_{—}631905 at the filing date of this patent application. While it is assumed that the sequence provided in database entry NP_{—}631905 is correct, it is noted that any corrected version of this sequence, in case said sequence turned out to be incorrect, is deliberately envisaged for the purposes of the present invention.

Arai et al. (2002) described the STARS protein as a novel, evolutionary conserved actin-binding protein, that is expressed specifically in cardiac and skeletal muscle cells. According to Arai et al. (2002), STARS binds to the I-band of the sarcomere and to actin filaments in transfected cells, where it activates Rho-signalling events.

In 2005, the same group showed that STARS activates serum response factor (SRF) by inducing nuclear translocation of myocardin-related transcription factors (MRTFs) (Kuwahara et al. (2005)). The authors conclude that STARS is involved in a muscle-specific mechanism.

The term “polynucleotide” is understood to comprise polydeoxyribonucleotides as well as polyribonucleotides. In other words, all forms of DNA, e.g. genomic DNA and cDNA, and RNA, e.g. mRNA, tRNA, rRNA and genomic RNA (such as in case of RNA viruses) are embraced.

The polynucleotides of (b) include polynucleotides encoding polypeptides comprising or consisting of fragments of the amino acid sequence set forth in SEQ ID NO: 2. It is well known in the art that functional polypeptides may be cleaved to yield fragments with unaltered or substantially unaltered function. Such cleavage may include the removal of a given number of N- and/or C-terminal amino acids. Additionally or alternatively, a number of internal (non-terminal) amino acids may be removed, provided the obtained polypeptide has arteriogenic activity. Said number of amino acids to be removed from the termini and/or internal regions may be one, two, three, four, five, six, seven, eight, nine, ten, 15, 20, 25, 30, 40, 50 or more than 50. Any other number between one and 50 is also deliberately envisaged. In particular, removals of amino acids which preserve sequence and boundaries of any conserved functional domain(s) or subsequences in the sequence of SEQ ID NO: 2 are particularly envisaged. Means and methods for determining such domains are well known in the art and include experimental and bioinformatic means. Experimental means include the systematic generation of deletion mutants and their assessment in assays for arteriogenic activity known in the art and as described in the Examples enclosed herewith (see above and Example 3 enclosed herewith). Bioinformatic means include database searches. Suitable databases included protein sequence databases. In this case a multiple sequence alignment of significant hits is indicative of domain boundaries, wherein the domain(s) is/are comprised of the/those subsequences exhibiting an elevated level of sequence conservation as compared to the remainder of the sequence. Further suitable databases include databases of statistical models of conserved protein domains such as Pfam maintained by the Sanger Institute, UK (www.sanger.ac.uk/Software/Pfam).

The polynucleotides of (c) include polynucleotides which hybridize with the polynucleotides of (a) or (b). It is well known in the art how to perform hybridization experiments with nucleic acid molecules and polynucleotides, i.e. the person skilled in the art knows what hybridization conditions s/he has to use in accordance with the present invention. Such hybridization conditions are referred to in standard text books such as “Molecular Cloning A Laboratory Manual”, Cold Spring Harbor Laboratory (1989) N.Y. or Higgins, S. J., Hames, D. “RNA Processing: A practical approach”, Oxford University Press (1994), Vol. 1 and 2.

“Stringent hybridization conditions” refers to conditions which comprise, e.g. an overnight incubation at 65° C. in 4×SSC (600 mM NaCl, 60 mM sodium citrate) followed by washing at 65° C. in 0.1×SSC for one hour. Alternatively, hybridization conditions can comprise: an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Said conditions for hybridization are also known by a person skilled in the art as “high stringent conditions for hybridization”. Also contemplated are polynucleotides according to (c) that hybridize to the polynucleotides of (a) or (b) at lower stringency hybridization conditions (“low stringent conditions for hybridization”). Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 50° C. in 4×SSC or an overnight incubation at 37° C. in a solution comprising 6×SSPE (20×SSPE=3M NaCl; 0.2M NaH₂PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The polynucleotides according to (c) furthermore comprise polynucleotides comprising or consisting of a nucleotide sequence encoding a polypeptide, wherein said polypeptide exhibits at least 75%, 80%, 85%, 90%, 95%, 96%, 97%, 98% or 99% sequence identity with the sequence of the polypeptide encoded by the polynucleotide of (a) or (b) over the entire length, and wherein said polypeptide has arteriogenic activity.

Two nucleotide or protein sequences can be aligned electronically using suitable computer programs known in the art. Such programs comprise BLAST (Altschul et al. (1990), J. Mol. Biol. 215, 403-410), variants thereof such as WU-BLAST (Altschul & Gish (1996), Methods Enzymol. 266, 460-480), FASTA (Pearson & Lipman (1988), Proc. Natl. Acad. Sci. USA 85, 2444-2448) or implementations of the Smith-Waterman algorithm (SSEARCH, Smith & Waterman (1981), J. Mol. Biol. 147, 195-197). These programs, in addition to providing a pairwise sequence alignment, also report the sequence identity level (usually in percent identity) and the probability for the occurrence of the alignment by chance (P-value). Programs such as CLUSTALW (Higgins et al. (1994), Nucleic Acids Res. 22, 4673-4680) can be used to align more than two sequences.

Said polynucleotides include polynucleotides encoding fusion proteins. Consequently, said polypeptides include polypeptides which are fusion proteins. Those components of said fusion proteins, which are not ABRA/STARS sequences or fragments or variants thereof as defined herein above, include amino acid sequence which confer desired properties such as modified/enhanced stability, modified/enhanced solubility and/or the ability of targeting one or more specific cell types. For example, fusion proteins with antibodies specific for cell surface markers or with antigen-recognizing fragments of said antibodies are envisaged.

Polypeptides comprised in the pharmaceutical composition of the invention include polypeptides wherein one or more amino acids are chemically modified whilst maintaining arteriogenic activity.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of a pharmaceutical composition administered to an individual will, inter alia, depend on the nature of the compound. For example, if said compound is a polypeptide or protein, the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. If given continuously, the pharmaceutical composition is typically administered at a dose rate of about 1 μg/kg/hour to about 50 μg/kg/hour, either by 14 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, rectally, parenterally, intracisternally, intravaginally, intraperitoneally, topically (as by powders, ointments, drops or transdermal patch), bucally, or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12.98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

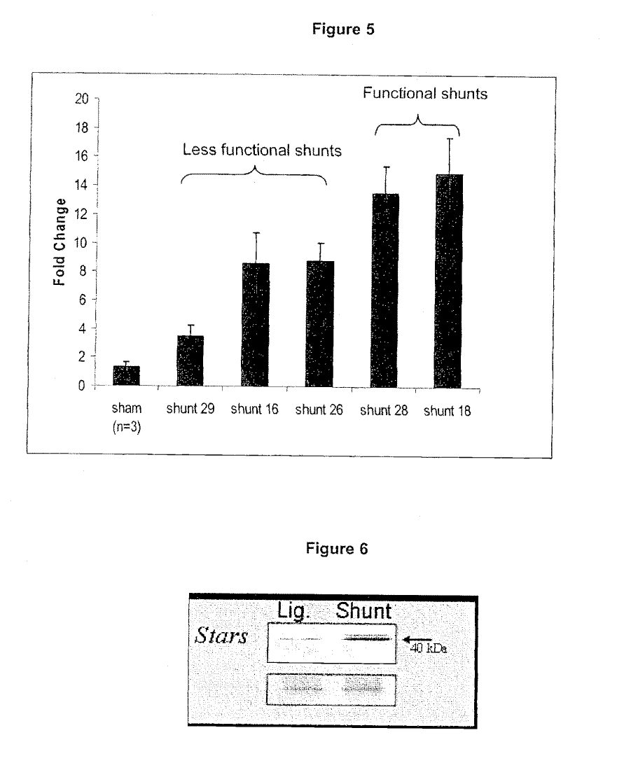

The present inventors surprisingly discovered that ABRA/STARS functions as a master regulator of arteriogenesis (see Examples 4 to 9 and FIGS. 5, 6, 7, 10, 11, 12, 13 and 14). This is unexpected, noting that the prior art reviewed herein above consistently reports an involvement of ABRA/STARS in skeletal and cardiac muscle-specific mechanisms.

The present invention furthermore relates to the use of (a) a polynucleotide comprising or consisting of a nucleotide sequence encoding a polypeptide with the amino acid sequence as set forth in SEQ ID NO: 2; (b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity; (c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or (d) a polypeptide encoded by the polynucleotide of any one of (a) to (c) for the preparation of a pharmaceutical composition for the treatment or prevention of an ischemic condition and/or for the treatment of a patient undergoing tissue engineering.

The term “ischemic” means “affected by ischemia”, wherein “ischemia” designates a low oxygen state, for example due to obstruction of the arterial blood supply or inadequate blood flow leading to hypoxia in the tissue. Preferred ischemic conditions according to the invention are detailed further below.

The term “tissue engineering” designates the development of biological substitutes that restore, maintain or improve tissue function. The aims of tissue engineering include the production of functional replacement tissue for clinical use. Tissue engineering may take place in vivo, i.e., tissue is engineered or grown in a patient. It is known (L'heureux et al. (2006)) that during the course of tissue engineering frequently a shortage of blood supply arises. As a consequence, patients undergoing tissue engineering or receiving engineered tissue are in need of a stimulation of arteriogenesis in the region(s) to be engineered. Preferred tissue to be engineered include blood vessels including arteries. Alternatively, and as detailed further below, tissue engineering may take place in vitro or ex vivo.

In a preferred embodiment of the pharmaceutical composition or the use according to the invention, said polypeptide exhibits at least 80% sequence identity. In a further preferred embodiment of the pharmaceutical composition or the use according to the invention, said polypeptide exhibits at least 90% sequence identity.

In a further preferred embodiment of the pharmaceutical composition or the use according to the invention, said nucleotide sequence has the sequence set forth in SEQ ID NO: 1.

The sequence of SEQ ID NO: 1 is the sequence of the mRNA for the human ABRA protein. The sequence can be found, for example, in the Entrez Nucleotide database maintained by the National Center for Biotechnology Information (NCBI) under the database accession number NM_{—}139166. The sequence of SEQ ID NO: 1 is identical with the sequence provided in database entry NM_{—}139166 at the filing date of this patent application. While it is assumed that the sequence provided in database entry NM_{—}139166 is correct, it is noted that any corrected version of this sequence, in case said sequence turned out to be incorrect, is deliberately envisaged for the purposes of the present invention.

Also included within the scope of the invention are polynucleotides which differ from the sequence set forth in SEQ ID NO: 1 as a consequence of the degeneracy of the genetic code, i.e., the envisaged polynucleotides, while different in sequence from the sequence set forth in SEQ ID NO 1, encode the same polypeptide as does the sequence set forth in SEQ ID NO: 1.

In a further preferred embodiment of the pharmaceutical composition or the use according to the invention, said nucleotide sequence is comprised in a vector.

The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector of the present invention may, in addition to the nucleotide sequences of the invention, comprise expression control elements, allowing proper expression of the coding regions in suitable hosts. Such control elements are known to the artisan and may include a promoter, a splice cassette, translation initiation codon, translation and insertion site for introducing an insert into the vector. Preferably, the nucleotide sequence of the invention is operably linked to said expression control sequences allowing expression in eukaryotic or prokaryotic cells.

Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook (1989), loc. Cit., and Ausubel, Current Protocols in Molecular Biology, Green Publishing Associates and Wiley Interscience, N.Y. (1989), (1994). Alternatively, the nucleotide sequences and vectors of the invention can be reconstituted into liposomes for delivery to target cells. Thus, according to the invention relevant sequences can be transferred into expression vectors where expression of a particular polypeptide/protein is required. Typical cloning vectors include pBscpt sk, pGEM, pUC9, pBR322 and pGBT9. Typical expression vectors include pTRE, pCAL-n-EK, pESP-1, pOP13CAT.

The term “control sequence” or “control element” refers to regulatory DNA sequences which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the vector of the invention is preferably an expression vector. An “expression vector” is a construct that can be used to transform a selected host cell and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotic and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the P_{L}, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. In this context, suitable expression vectors are known in the art such as Okayama-Berg cDNA expression vector pcDV1 (Pharmacia), pCDM8, pRc/CMV, pcDNA1, pcDNA3 (In-vitrogene), pSPORT1 (GIBCO BRL). An alternative expression system which could be used is an insect system. In one such system, Autographa californica nuclear polyhedrosis virus (AcNPV) is used as a vector to express foreign genes in Spodoptera frugiperda cells or in Trichoplusia larvae. The coding sequence of a polynucleotide of the invention may be cloned into a nonessential region of the virus, such as the polyhedrin gene, and placed under control of the polyhedrin promoter. Successful insertion of said coding sequence will render the polyhedrin gene inactive and produce recombinant virus lacking coat protein. The recombinant viruses are then used to infect S. frugiperda cells or Trichoplusia larvae in which the protein of the invention is expressed (Smith, J. Virol. 46 (1983), 584; Engelhard, Proc. Nat. Acad. Sci. USA 91 (1994), 3224-3227).

In a preferred embodiment, said vector is comprised in a liposome. Liposome-mediated transfer of polynucleotides is well known in the art (see above).

In another preferred embodiment, said vector is an adenoviral vector, adeno-associated viral vector or lentiviral vector. Adeno-associated viral vectors and lentiviral vectors are particularly preferred in those cases where the patient recently went through an infection by an adenovirus. In such a case the presence of antibodies directed against said adenovirus may prevent success of a therapy using the adenoviral vector.

In a more preferred embodiment, said pharmaceutical composition comprising a vector is to be delivered by means of a catheter to be inserted into the vessel to be treated.

In another more preferred embodiment, said pharmaceutical composition comprising a polypeptide is to be administered by injection at or near the site to be treated.

In a further preferred embodiment, said nucleotide sequence has been transfected into a cell obtained from a patient to be treated. Preferably, said cells are blood cells obtained from a peripheral vein of the patient to be treated. The cells obtained from the patient may be cultured and propagated in vitro prior to transfection. Preferred cells to be obtained and transfected are monocytes of the peripheral blood. Monocytes are amenable to infection by viral vectors and exhibit advantageous homing properties. More specifically, monocytes are capable of attaching to growing collateral vessels (Schaper et al. (1976), WO 00/60054). Upon transfection, the cells are to be re-introduced into the patient. Re-introducing can be done by injection into or near the collateral vessel(s). Alternatively, simple intravenous injection may be performed. In this case, a co-transfection with Mac-1 cDNA is preferred. Expression of the Mac-1 gene in the transfected cells ensures that the transfected cells bind to docking sites in the collateral vessel(s).

In a further preferred embodiment of the uses according to the invention, said ischemic condition is a consequence of arterial injury, arterial degeneration, arterial occlusion or arterial re-occlusion.

Preferably, said ischemic condition is selected from atherosclerosis, angina pectoris, infarct, heart attack, stroke, renal infarct, hypertension, wounds, peripheral ischemic vascular disease, bone fracture and abortus imminens. Wounds include wounds which involve arterial lesions. Other ischemic conditions, which are also envisaged, include ischemic conditions arising from diabetes or smoking.

In a further preferred embodiment of the uses according to the invention, the affected organs or body parts are selected from heart, brain, kidneys, limbs and blood vessels. Blood vessels include arteries and veins.

In a further preferred embodiment, said pharmaceutical composition comprises said nucleotide sequence and/or said polypeptide as the sole active agent(s).

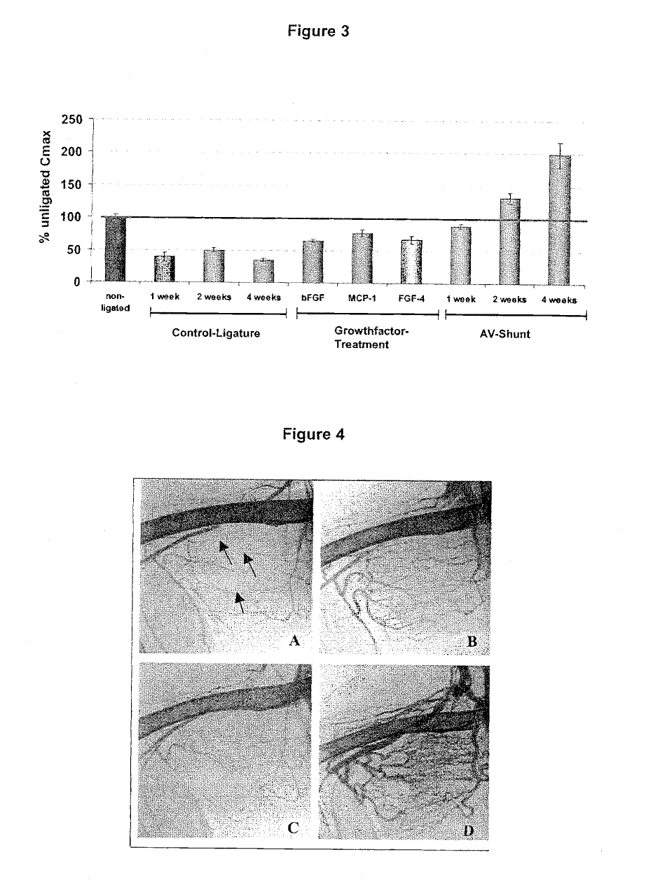

Another preferred embodiment relates to uses and pharmaceutical compositions, wherein said pharmaceutical compositions additionally comprise one or more angiogenic growth factor(s). Angiogenic growth factors have described in the art in their role as stimulators of angiogenesis, which, as described above, is to held distinct from arteriogenesis. As exemplified in Example 3 and FIG. 3, the present inventors could show that angiogenic growth factors exhibit a stimulatory effect also on arteriogenesis. While it is expected that angiogenic growth factors fail to elicit an effect comparable to ABRA/STARS polynucleotides or polypeptides, it is expected that their co-administration with said ABRA/STARS polynucleotides and/or polypeptides further enhances the stimulatory effect on arteriogenesis.

Preferably, said angiogenic growth factor is selected from the group consisting of FGF-2 (bFGF), FGF-4, MCP-1, PDGF and VEGF.

In a further preferred embodiment of the pharmaceutical composition or the use of the invention, said pharmaceutical composition comprises a stent. The term “stent” is well known in the art and includes expandable wire meshes and hollow perforated tubes that are inserted into a hollow structure of the body to keep it open. Said hollow structure includes blood vessels which in turn include arteries. The pharmaceutical composition of the invention comprising a stent may be a stent coated with the polypeptide and/or the polynucleotide of the main embodiment. Any constituent of a pharmaceutical composition of the invention (besides the stent itself) may be comprised in a coating layer of a stent according to the invention.

The present invention furthermore relates to an in vitro or ex vivo method of tissue engineering, wherein the method comprises bringing the tissue into contact with (a) a polynucleotide comprising or consisting of a nucleotide sequence encoding a polypeptide with the amino acid sequence as set forth in SEQ ID NO: 2; (b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity; (c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or (d) a polypeptide encoded by the polynucleotide of any one of (a) to (c). This embodiment relates to the filed of regenerative medicine and pertains to growth and/or modification of tissue outside the human or animal body. As noted above, conditions of limiting blood supply may arise during tissue engineering. As a consequence, stimulation of arteriogenesis in the tissue is desirable. Preferred tissue to be engineered include blood vessels including arteries.

Another subject of the present invention is a method of identifying modulators of the biological activity of the polynucleotide or the polypeptide as defined in claim 1, comprising the following steps: (a) bringing into contact said polynucleotide or polypeptide with a test compound; and (b) comparing the biological activity in the presence of said test compound with the biological activity in the absence of said test compound, wherein the biological activity in the presence of a modulator and in the absence thereof differ. This embodiment relates to a screening method for compounds acting as modulators. Step (a) is effected under conditions which allow the test compound to interact physically and/or functionally with said polynucleotide or polypeptide. Suitable conditions depend on the test compounds. The skilled person is well aware of such suitable conditions which include, for example, buffered solutions comprising the polynucleotide or polypeptide, the test compound(s) and, if required, any further factors necessary for function and integrity of said polynucleotide or polypeptide and/or of said test compound(s).

Preferably, said method is effected in high-throughput format. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to said activity.

Preferably, said modulator is an activator. The term “activator” according to the invention designates a test compound which increases the biological activity of said polynucleotide or polypeptide by 10%, 20%, 30%, 40% or 50%, more preferred by 60%, 70%, 80%, 90% or 100%, and yet more preferred by 200%, 500%, 1000% or more. Preferably, said biological activity is the expression of said polynucleotide and/or the stimulation of arteriogenesis.

In a preferred embodiment, the method comprises, after step (a) and prior to step (b) the further step of (a′) determining whether said test compound binds to said polynucleotide or polypeptide. The determination of binding test compounds in step (a′) relates to any binding assay, preferably biophysical binding assay, which may be used to identify binding test molecules prior to performing the functional/activity assay with the binding test molecules only. Suitable biophysical binding assays are known in the art and comprise fluorescence polarization (FP) assay, fluorescence resonance energy transfer (FRET) assay and surface plasmon resonance (SPR) assay. Step (a′) is particularly advantageous if said binding assay is more amenable to high throughput than the functional assay. Preferably, only test compounds exhibiting binding affinity to said polynucleotide or polypeptide are fed into step (b) of comparing biological activity in presence and absence of the test compound.

In a further preferred embodiment of the method of identifying modulators, said polynucleotide and/or said polypeptide is/are comprised in a cell transfected with said polynucleotide. This embodiment relates to a cellular screen. In a cellular screen modulators may be identified which exert their modulatory activity by physically interacting with the target molecule (said polynucleotide or said polypeptide), or alternatively (or additionally) by functionally interacting with said target molecule, i.e., by interfering with the pathway(s) present in the cells employed in the cellular assay and in control of the biological activity of said polynucleotide or said polypeptide.

In another preferred embodiment of the method of identifying modulators of the invention, the method comprises the further step of (c) formulating one or more modulators obtained with a pharmaceutically acceptable carrier, excipient or diluent.

In a more preferred embodiment, the method of identifying modulators comprising, after step (b) and prior to step (c) the step of (b′) optimizing the pharmacological properties of the modulator.

Methods for the optimization of the pharmacological properties of compounds identified in screens, generally referred to as lead compounds, are known in the art and comprise a method of modifying a compound identified as a lead compound to achieve: (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carboxylic acids, or (iii) esterification of hydroxyl groups to, e.g. phosphates, pyrophosphates or sulfates or hemi-succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiffs bases, oximes, acetates, ketales, enolesters, oxazolidines, thiazolidines or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The present invention also relates to an antibody identified or obtainable by said method of identifying modulators. Preferably said antibody is an antibody capable of binding the polypeptide defined in the main embodiment. More preferably, said binding is specific. Methods for raising antibodies against a given polypeptide or protein are well know in the art.

The term “antibody” includes monoclonal antibodies, polyclonal antibodies, single chain antibodies, or fragments thereof that specifically bind said polypeptide, also including bispecific antibodies, synthetic antibodies, antibody fragments, such as Fab, a F(ab₂)′, Fv or scFv fragments etc., or a chemically modified derivative of any of these. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit.

The term “monoclonal” or “polyclonal antibody” (see Harlow and Lane, (1988), loc. cit.) also relates to derivatives of said antibodies which retain or essentially retain their binding specificity. Preferred derivatives of such antibodies are chimeric antibodies comprising, for example, a mouse or rat variable region and a human constant region.

The term “scFv fragment” (single-chain Fv fragment) is well understood in the art and preferred due to its small size and the possibility to recombinantly produce such fragments.

The term “specifically binds” in connection with the antibody used in accordance with the present invention means that the antibody etc. does not or essentially does not cross-react with polypeptides of similar structures. Cross-reactivity of a panel of antibodies etc. under investigation may be tested, for example, by assessing binding of said panel of antibodies etc. under conventional conditions (see, e.g., Harlow and Lane, (1988), loc. cit.) to the polypeptide of interest as well as to a number of more or less (structurally and/or functionally) closely related polypeptides. Only those antibodies that bind to the polypeptide/protein of interest but do not or do not essentially bind to any of the other polypeptides which are preferably expressed by the same tissue as the polypeptide of interest, are considered specific for the polypeptide/protein of interest.

In a particularly preferred embodiment of the method of the invention, said antibody or antibody binding portion is or is derived from a human antibody or a humanized antibody.

The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861.

Antibodies according to the invention which act as modulators of biological activity can be obtained or identified by subjecting antibodies to the method of identifying modulators according to the invention.

The Figures show:

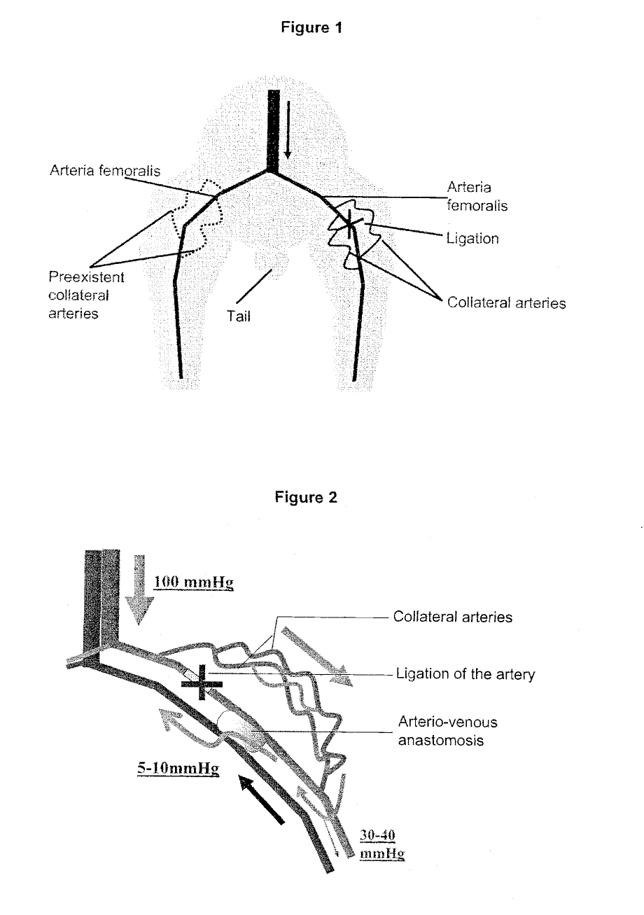

FIG. 1: Schematic illustration of the model of femoral artery ligation in the hindlimb of a rabbit. In the left limb the unoccluded arteria with pre-existent collateral arteries is shown. In the right limb the artery is occluded (ligation) and the collateral arteries enlarged to assure perfusion of the distal limb (arteriogenesis).

FIG. 2: Shown is a magnified part of the vascular system of one limb. The artery is ligated (Ligation) and the collateral arteries bypass this occlusion. Below the ligation an arterio-venous anastomosis is prepared, because of the low pressure in the vein (5-10 mmHg), in contrast to the higher pressure in the lower limb (30-40 mmHg) the main fraction of the blood flows out of the collaterals directly into the vein. This is associated with an increase in blood flow and fluid shear stress within the collateral arteries.

FIG. 3: Results of the measurements of maximal collateral conductances in % of Ccmax of unoccluded femoral arteries in contrast to growth factor treated and arterio-venous-(av-)shunt treated collateral systems. The aim to reach conductances of non-ligated femoral arteries was not reached in control-ligatures and after growth factor treatments. After one week shunt treatment this aim is reached, after two weeks it is surpassed and after 4 weeks the conductance is doubled in contrast to unligated arteries.

FIG. 4: Post mortem Angiographies of collateral systems after different treatments A) Ligature of the artery without any further treatment after 1 week: only small collaterals are visible (arrows) B) Ligature plus shunt treatment after 1 week: a lot of collaterals with a larger diameter are visible C) Ligature without any further treatment after 4 weeks: some collaterals are visible D) Ligature plus shunt treatment after 4 weeks: huge amount of large collateral arteries.

FIG. 5: ABRA/STARS (Abra/Stars) gene transcription: qRT PCR results for Abra/Stars mRNA transcription in rats 5 days after shunt surgery vs. sham operated animals (Fold Change=1). Note that functionality of shunt correlates with Abra/Stars transcription. The less functional (decreased shear stress) shunts 29, 16 and 26 show also decreased Abra/Stars transcription. The highly functional shunts 28 and 18 induce high Abra/Stars transcription.

FIG. 6: Abra/Stars Protein expression: Western blot analysis of Abra/Stars in 7 day of shunt treatment collaterals vs. ligation controls.

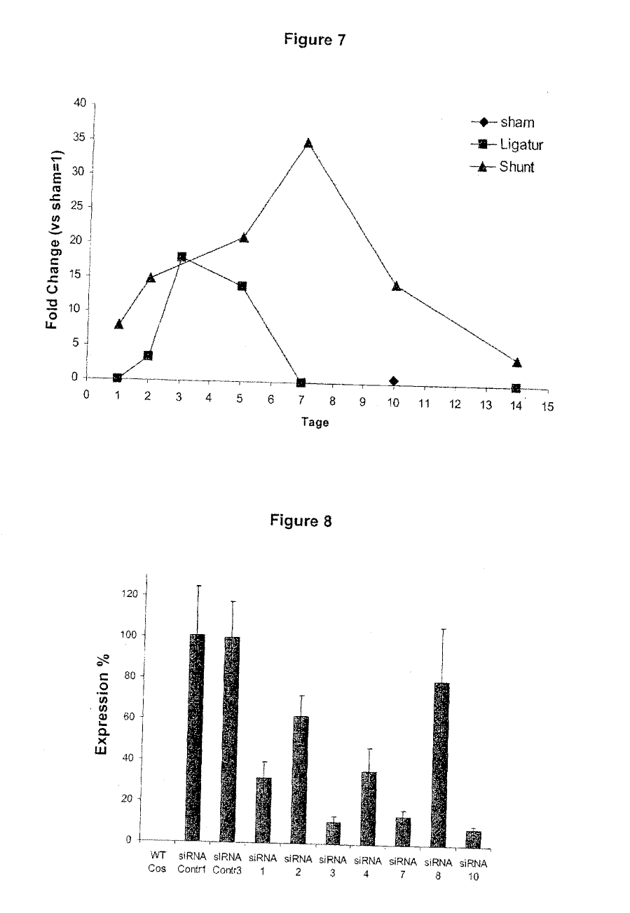

FIG. 7: Time course of Abra/Stars transcription in rat shunts vs ligation. “Shunt transcription” of Abra/Stars peaks around day 7 with a fold change of 35 and does not level off after 14 days. On the side of ligation Abra/Stars transcription peaks already around day 3 and completely disappears after 7 days.

FIG. 8: Co-transfection of Abra/Stars expression- and siRNA Abra/Stars-vectors in COS1 cells. Note that there is no endogenous expression of Abra/Stars in this cell type. More than 80% down regulation of Abra/Stars was achieved with three siRNAs (siRNA 3, 7 and 10). As controls COS cells were co-transcfected with Abra/Stars expression vector and nonsense siRNA expressing Plasmid (Contr1 an 2). Abra/Stars transcription of Contr1 and 2 was considered 100%.

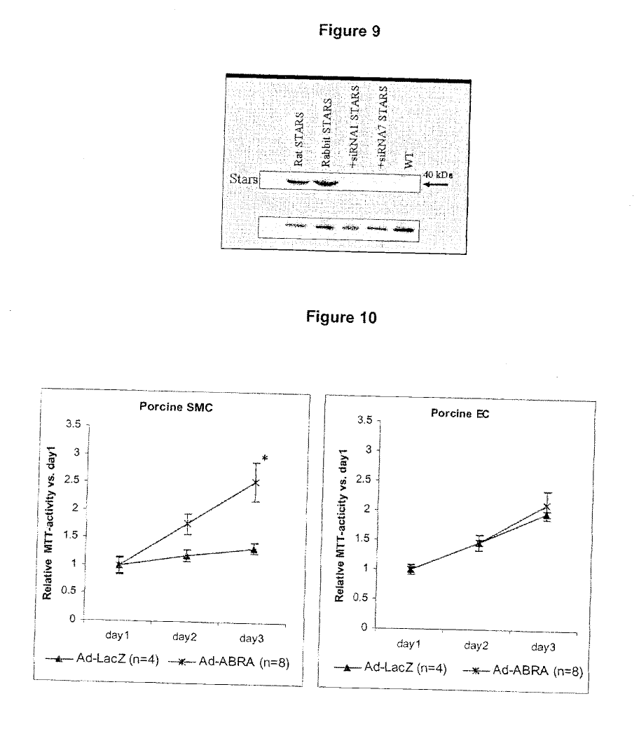

FIG. 9: Western blot of siRNA mediated knock down of Abra/Stars in transfected COS1 cells. The 2 tested siRNAs (siRNA 1 and 7) are functional in mediating a protein knock down below the detection limit of the Western blot. Note that the antibody developed for rat Abra/Stars also detects its rabbit homologue.

FIG. 10: Proliferation activity of ABRA-transfected porcine SMC (a) and EC (b) cells. To monitor cell proliferation activity the MTT Cell Proliferation Assay (ATCC™) was used. Proliferation activity is shown as relative change of formazan absorbance at 570 nm compared to day1. (*p<0.05)

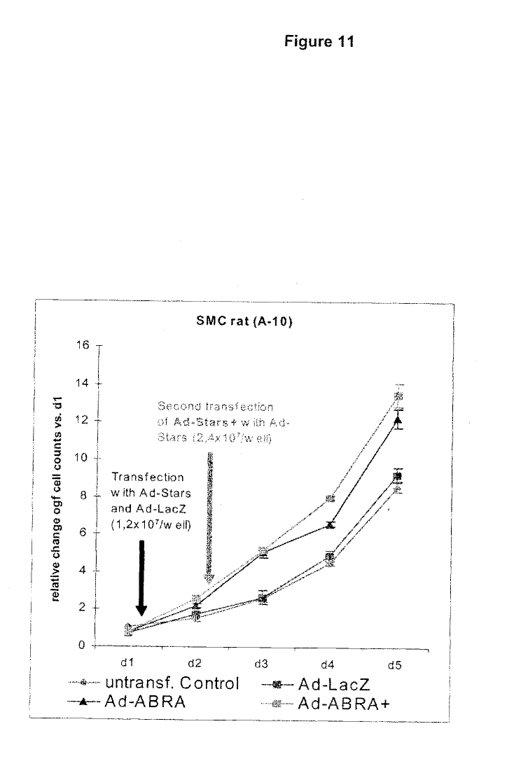

FIG. 11: Proliferation of Ad-ABRA transfected rat SMC (A-10). Cells were transfected using recombinant adenovirus Ad-ABRA and Ad-LacZ (3.75×10⁷ infectious units/well) on day one (d1). On day two (d2) one set of ABRA-transfected cells were transfected again with Ad-ABRA (Ad-ABRA+).

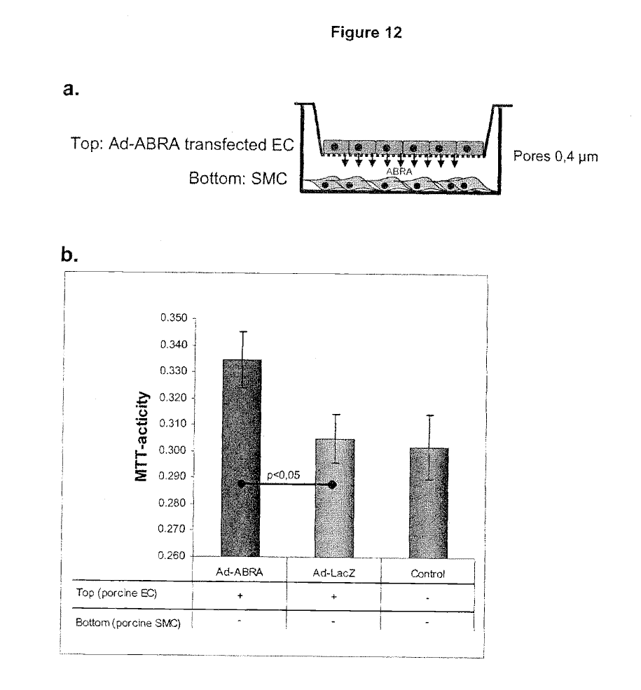

FIG. 12: Cell culture secretion experiment with porcine SMC an EC. (a) Ad-ABRA transfected cells were plated on top (ThinCert™ inserts, pore size 0.4 μm, Greiner bio-one) of porcine SMC (bottom). Secreted small molecules are transported through the membrane. (b) Proliferation activity (MTT Cell Proliferation Assay, ATCC®) of SMC was measured on day 2 and 3 after transfection.

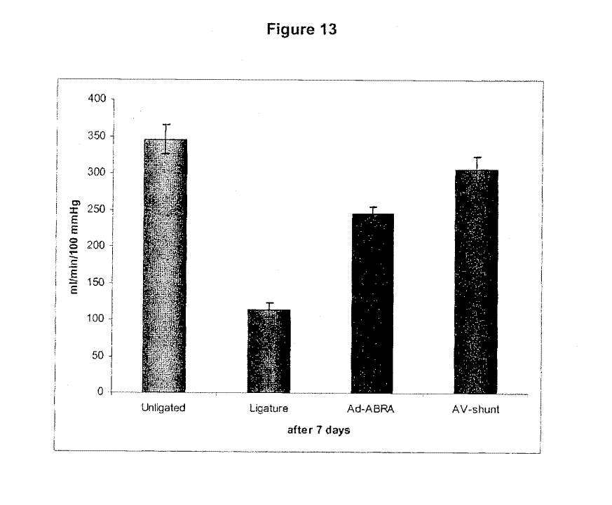

FIG. 13: Results of the measurements of maximal collateral conductance in % of CCmax of unoccluded femoral arteries (Unligated) in contrast to ligated (Ligature), AV-shunt treated (AV-shunt) and ABRA transfected (Ad-ABRA) collateral systems. The aim to reach conductance of unligated femoral arteries was not reached in ligatures. After one week the conductance of ABRA transfected collaterals almost reaches AV-shunt values.

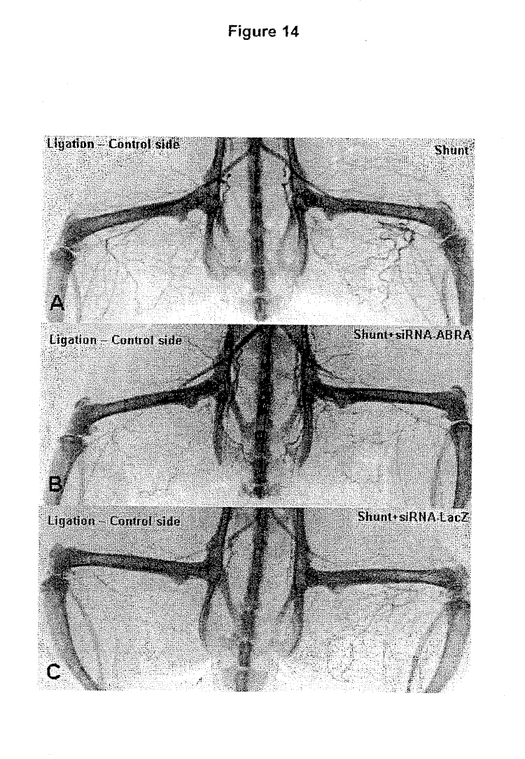

FIG. 14: Knock down of ABRA/STARS using siRNA in the hind limb ischemic model of rats upgraded with an arteriovenous shunt in the left leg. The right leg was just treated with a simple ligation and served as an intra-individual control. A. Shunt without siRNA treatment. B. Knock down of ABRA of the shunted side using local adenoviral transfection of siRNA-producing transgene. Collateral growth was inhibited and is comparable to the control side. C. Intra collateral transfection with a nonsense siRNA. Collateral growth is comparable to shunt-treatment without transfection (A).

The following examples illustrate the invention but should not be construed as being limiting.

### EXAMPLE 1

### General Approach for the Identification of Differentially Regulated Genes

To achieve a chronically high fluid shear stress we surgically connected the distal stump of the occluded artery, which collects most of the bypass-flow, with the accompanying vein so that a short-circuit existed in the sense that the collateral blood flow was diverted into the low-pressure, low resistance, venous system instead of flowing into the distal high resistance vascular bed. In this way a constantly high flow was created because of the large pressure gradient between artery and vein. Even though the collaterals grow, the fluid shear stress does not decrease, as the blood flow decreases with the 4^{th} power of the radius of the vessel, and the shear stress decreases only with the 3^{rd} power of the radius of the vessel.

The extra tissue growth of the “super-bypasses” was harvested, the RNA extracted and subjected to micro-array analysis from which hundreds of genes showed changes in their activity.

### EXAMPLE 2

### The Animal Model

We developed an animal model with sustained fluid shear stress within the collateral arteries, to circumstantiate that fluid shear stress is the driving force in arteriogenesis.

To induce arteriogenesis in rabbits, we ligated the femoral artery, which is the main artery of the hindlimb (FIG. 1). Next we established a direct connection between the artery distal to the ligation and the accompanying vein. Thereby a decrease of pressure in the collateral arteries and an increase in fluid shear stress was established (FIG. 2).

### EXAMPLE 3

### Maximum Collateral Conductance Measurements

Hemodynamic measurements were performed to quantify the maximum capacity of the collateral system on the basis of measuring the maximum collateral conductance (Ccmax). Methods of measuring the maximum collateral conductance are well known to the person skilled in the art and can be performed as outlined in the description and as described, for example, in Schaper et al. (1976) und Pasyk et al. (1982).

In particular, for the terminal experiment the animals were anesthetized again and both iliac arteries were exposed and outfitted with ultrasonic flow probes to measure blood flow in ml/min. These were positioned proximal to the deep femoral artery, i.e., the stem of most of the growing collateral vessels. A small PE 50 catheter was advanced through a side-branch into the abdominal aorta for the infusion of adenosine. A fluid filled catheter was introduced into the carotid artery for the measurement of systemic arterial pressure in mmHg. Both pedal arteries were prepared and catheters inserted for the measurement of the pressure gradient over the collateral network or along the intact femoral artery and its tributaries. Adenosine was infused at increasing concentrations until the central aortic pressure began to fall. This happened usually at a concentration of 600 micrograms/kg per minute. Pressure and flow signals were fed into an AD-converter and were digitally recorded on a MacLab computer. Maximal conductances (Cmax) were calculated by dividing maximal adenosine-recruited flows by the pressure gradient across the collateral network (ml/min/mmHg).

The aim was to reach maximum conductances comparable to those of unligated femoral arteries, that means to reach a complete restoration of arterial function. Before we established our shunt experiments, even after treatment with different growth factors, it was not possible to reach a full recovery. To reach a full recovery (FIG. 3) means we have been able to create “super-bypasses”. These “super-bypasses” are visible in post mortal angiographies (FIG. 4).

### EXAMPLE 4

### Differential Expression

To perform molecular biology experiments, we had to establish the shunt model in the rat. The rat angiographies showed, that shunt treatment stimulates arteriogenesis in rats to the same degree as in rabbits.

From dissected rat collateral arteries total RNA was isolated and subjected to micro array analysis (Rat whole Genome Array, Agilent). Among the highest significant up regulated targets we focussed on Abra/Stars (actin-binding Rho activating protein/striated activator of Rho-dependent signalling). Using quantitative PCR an up to 14-fold increase of Abra/Stars transcripts was confirmed 5 days after shunt surgery (FIG. 5). Note that variations of fold changes of Abra/Stars in “shunt” collaterals correlate with quality and functionality of performed shunt implementation in the animal. To confirm up regulation of Abra/Stars also on the protein level we conducted Western blot analysis of extracted proteins from “shunt” and control collaterals (FIG. 6).

To investigate the time course of Abra/Stars transcription, total RNA from collateral arteries was isolated at different time points after shunt implementation and subjected to qRT PCR analysis (FIG. 7). Whereas in control collaterals (ligation without shunt) the transcription peaks at day 3 and completely disappears after seven days, in “shunt” collaterals Abra/Stars is transcribed at much higher levels and still significantly upregulated after 14 days. Thus Abra/Stars expression is closely linked with shear stress levels in shunt operated rats.

### EXAMPLE 5

### Overexpression and Knock-Down

To prove that Abra/Stars is a key regulator in arteriogenesis overexpressing and knock down vectors were produced. To overexpress Abra/Stars the rat and rabbit open reading frame was cloned separately into a mammalian expression vector (pcDNA3neo, Invitrogen). To reduce Abra/Stars transcripts we applied siRNA mediated knock down. This was achieved by cloning Abra/Stars target sequences into an siRNA transcription system (pSilencer Neo, Ambion). These vectors were first tested in transfected COS1 cells. As in this cell type no endogenous expression of Abra/Stars is observed, cells were co-transfected with both vectors. Using nonsense siRNAs as controls we identified three functional siRNAs leading to more than 80% knock down of Abra/Stars transcripts (FIG. 8). This result was also confirmed by Western blot analysis (FIG. 9), where Abra/Stars proteins was knocked down below the detection limit of the antibody.

In order to test Abra/Stars knock down in the shunt model in vivo we constructed recombinant siRNA adenovirus and performed a local intracollateral virus transduction of shunt treated rats (FIG. 14). Knock down of ABRA leads to decreased collateral formation (FIG. 14b) compared to shunt-treated (FIG. 14a) and control virus treated rats (nonsense siRNA) (FIG. 14 c).

### EXAMPLE 6

### ABRA/STARS-expressing Porcine SMC Proliferate—EC do not

Using the MTT Cell Proliferation Assay (ATCC®) we could show that ABRA increases proliferation activity of isolated porcine smooth muscle cells (SMC) (FIG. 10a). In contrast porcine endothelial cells (EC) in culture did not show changes in proliferation activity after ABRA transfection compared to LacZ controls (FIG. 10b).

The yellow tetrazolium (MTT; (3-(4,5-dimethylthiazol-2-yl)-2,5-diphenyltetrazoliumbromid)) is reduced by metabolic active cells, in part by the action of dehydrogenase enzymes, to generate reducing equivalents such as NADH and NADPH. The resulting intracellular formazan can be quantified by spectrophotometric means and illustrates cell proliferation.

Cells were plated on 96-well plate and transfected with ABRA and LacZ using recombinant adenovirus (1.175×10⁷ infectious units/well). On day 1, 2 and 3 10 ul MTT reagent was added to each well and incubated for 4 h. Then 100 ul of Detergent reagent was added and incubated in the dark at RT for 2 h. Absorbance was measured at 570 nm.

### EXAMPLE 7

### ABRA/STARS-expressing rat SMC Line Showed Increased Proliferation

In a second experiment cultured A-10 cells established from SMC of the thoracic aorta of a DB1X embryonic rats responded closely to ABRA-transfection with increased cell proliferation.

Cells were cultured on 24-well plates (5×10³/well) and transfected using recombinant adenovirus Ad-ABRA and Ad-LacZ (3.75×10⁷ infectious units/well). On day 2 one set of initially ABRA transfected cells were transfected again with recombinant ABRA-adenovirus (2.4×10⁷ infectious units/well). For 5 days duplicate wells (n=4) were harvested and counted to monitor cell proliferation.

After 2 days ABRA-transfected cells showed a doubled (92%) proliferation rate (FIG. 11). Those cells that were transfected again with ABRA on day 2 showed 21% more cells on day 3 compared to “normal” ABRA-transfected cells and a 60% increase of cell number compared to LacZ controls.

### EXAMPLE 8

### ABRA/STARS-transfected EC Secrete ABRA/STARS

In a cell culture secretion experiment porcine SMC were cultured in a 24-well plate. Using cell culture inserts (ThinCert™, Greiner bio-one) with a pore size of 0.4 μm (viruses are not transported through the membrane) Ad-ABRA transfected porcine EC cells were plated on top of each well (FIG. 12a). Proliferation activity (MTT Cell Proliferation Assay, ATCC®) was measured on day 2 and 3 after transfection. Porcine SMC show a significant increase in proliferation activity compared to Ad-LacZ controls (FIG. 12b). We therefore conclude that ABRA itself or ABRA-induced paracrine secretion by the EC into the medium leads to cell proliferation of SMC.

### EXAMPLE 9

### Intra-Collateral Adenoviral Gene Transfer of ABRA/STARS Improves Collateral Conductance

Using an intra-collateral adenoviral based gene transfer of ABRA we could substitute the effect of fluid shear stress (FSS). After ligation of the femoral artery in Rabbits we could induce collateral growth up to 90% compared to untransfected rabbits, with just a ligated femoral artery (see FIG. 13).

### CONCLUSION

In a hind limb ischemia model with an arterio-venous anastomosis (shunt) leading to chronically elevated fluid shear stress (FSS), we could show that this physical force markedly triggers collateral growth. For the first time, it was possible to completely compensate blood flow deficits.

The examples demonstrate that Abra/Stars expression is closely related to arteriogenesis. The transcription level as well as the time course of expression correlates strongly with collateral growth and makes Abra/Stars one of the key regulators of arteriogenesis.

Examples 6 to 9 using cultured cells demonstrate that ABRA/STARS acts as a mitogene for SMC, whereas EC are not affected and show no increased proliferation activity. On the other hand ABRA/STARS itself or ABRA/STARS induced paracrine mechanisms of Ad-ABRA transfected EC transduce signals that lead to increased proliferation activity of nearby SMC.

Finally we could show that intra collateral gene transfer using Ad-ABRA improves collateral conductance and growth (90%) after ligation of the femoral artery in rabbits compared to the untransfected but ligated control side.

On the other hand, ABRA knock down using siRNA in the shunt model of rat completely abolished the “shunt effect” and collateral remodelling was comparable to that seen in simple ligation controls.

So far ABRA has only been considered in the context of striated muscle cell differentiation (Arai et al., 2002; Kuwahara K et al., 2005). For the first time we could show that ABRA is also expressed highly in growing collaterals and plays a crucial role in collateral vessel growth triggered by increased FFS. As the shunt model leads to a complete restoration of vascular function (>100%) following occlusion of arteries we consider ABRA as one of the most promising targets for drug development in the context of vascular and cardiovascular disease.

### FURTHER REFERENCES

Arai et al. (2002). J. Biol. Chem. 277, 24453-24459.
Kuwahara et al. (2005). Mol. Cell. Biol. 25, 3173-3181.
L'heureux et al. (2006). Nat. Med. Feb. 19 (electronic publication ahead of print).
Pasyk et al. (1982). Am. J. Physiol. 242, H1031-1037.
Pipp et al. (2004). Arterioscler. Thromb. Vasc. Biol. 24, 1664-1668.
Schaper, J. et al. (1972). Am. J. Cardiol. 29, 851-859.
Schaper, J. et al., (1976). Virchows Arch. A. Pathol. Anat. Histol. 370, 193-205.
Schaper, W. et al. (1969). Cardiovasc. Res. 3, 315-323.
Schaper, W. (1971). The Collateral Circulation of the Heart. New York: American Elsevier Publishing Company. ISBN 0-7204-7300-4.
Schaper, W. et al. (1971). Circ. Res. 28, 671-679.

1. A pharmaceutical composition comprising
(a) a polynucleotide comprising or consisting of a nucleotide sequence encoding a polypeptide with the amino acid sequence as set forth in SEQ ID NO: 2;
(b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity;
(c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or
(d) a polypeptide encoded by the polynucleotide of any one of (a) to (c).

2. Use of
(a) a polynucleotide comprising or consisting of a nucleotide sequence encoding a polypeptide with the amino acid sequence as set forth in SEQ ID NO: 2;
(b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity;
(c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or
(d) a polypeptide encoded by the polynucleotide of any one of (a) to (c)
for the preparation of a pharmaceutical composition for the treatment or prevention of an ischemic condition and/or for the treatment of a patient undergoing tissue engineering.

3. The pharmaceutical composition of claim 1(c) or the use of claim 2(c), wherein said polypeptide exhibits at least 80% sequence identity.

4. The pharmaceutical composition of claim 1(c) or the use of claim 2(c), wherein said polypeptide exhibits at least 90% sequence identity.

5. The pharmaceutical composition of claim 1, 3 or 4 or the use of any one of claims 2 to 4, wherein said nucleotide sequence has the sequence set forth in SEQ ID NO: 1.

6. The pharmaceutical composition of claim 1 or 3 to 5 or the use of any of claims 2 to 5, wherein said nucleotide sequence is comprised in a vector.

7. The pharmaceutical composition or use of claim 6, wherein said vector is comprised in a liposome.

8. The pharmaceutical composition or use of claim 6, wherein said vector is an adenoviral vector, adeno-associated viral vector or lentiviral vector.

9. The pharmaceutical composition of claim 1 or 3 to 6 or the use of any of claims 2 to 6, wherein said nucleotide sequence has been transfected into a cell obtained from a patient to be treated.

10. The use of any one of claims 2 to 9, wherein said ischemic condition is a consequence of arterial injury, arterial degeneration, arterial occlusion or arterial re-occlusion.

11. The use of any one of claims 2 to 10, wherein said ischemic condition is selected from atherosclerosis, angina pectoris, infarct, heart attack, stroke, renal infarct, hypertension, wounds, peripheral ischemic vascular disease, bone fracture and abortus imminens.

12. The use of any one of claims 2 to 11, wherein the affected organs or body parts are selected from heart, brain, kidneys, limbs and blood vessels.

13. The pharmaceutical composition of any one of claim 1 or 3 to 9 or the use of any one of claims 2 to 12, wherein said pharmaceutical composition comprises said nucleotide sequence and/or said polypeptide as the sole active agent(s).

14. The pharmaceutical composition of any one of claim 1 or 3 to 9 or the use of any one of claims 2 to 12, wherein said pharmaceutical composition additionally comprises one or more angiogenic growth factor(s).

15. The pharmaceutical composition or the use of claim 14, wherein said angiogenic growth factor is selected from the group consisting of FGF-2 (bFGF), FGF-4, MCP-1, PDGF and VEGF.

16. The pharmaceutical composition of any one of claims 1, 3 to 9 or 13 to 15 or the use of any one of claims 2 to 15, wherein said pharmaceutical composition comprises a stent.

17. An in vitro or ex vivo method of tissue engineering, wherein the method comprises bringing the tissue into contact with
(a) a polynucleotide comprising or consisting of a nucleotide sequence encoding a polypeptide with the amino acid sequence as set forth in SEQ ID NO: 2;
(b) a polynucleotide comprising or consisting of a nucleotide sequence which is a fragment of a nucleotide sequence encoding the polypeptide with the amino acid sequence as set forth in SEQ ID NO: 2, wherein the polypeptide encoded by said polynucleotide has arteriogenic activity;
(c) a polynucleotide comprising or consisting of a nucleotide sequence encoding a polypeptide, wherein said polypeptide exhibits at least 70% sequence identity with the sequence of the polypeptide encoded by the polynucleotide of (a) or (b) over the entire length, and wherein said polypeptide has arteriogenic activity; and/or
(d) a polypeptide encoded by the polynucleotide of any one of (a) to (c).

18. A method of identifying modulators of the biological activity of the polynucleotide or the polypeptide as defined in claim 1, comprising the following steps:
(a) bringing into contact said polynucleotide or polypeptide with a test compound; and
(b) comparing the biological activity in the presence of said test compound with the biological activity in the absence of said test compound,
wherein the biological activity in the presence of a modulator and in the to absence thereof differ.

19. The method of claim 18, wherein said modulator is an activator.

20. The method of claim 18 or 19, wherein said biological activity is the expression of said polynucleotide and/or the stimulation of arteriogenesis.

21. An antibody identified or obtainable by the method of any one of claims 18 to 20.


SEQUENCE LISTING (2 sequences)

SEQ ID NO: 1 - DNA, 1322 bp, Homo sapiens
acacactcattttagccagtgtccaggctatcagcagagaaagacaggtgggcagcatggctccgggcgaaaaggaaagtggggagggcccagccaagagcgccctccggaagatacgcacagccaccctggtcatcagcttggcccgaggttggcagcagtgggcgaatgagaacagcatcaggcaggcccaggagcctacaggctggctgccgggagggacccaggactcacctcaagctcctaaaccaatcacaccccctacttcacaccagaaagctcagagtgccccaaagtcgccaccccgcctgccagaaggacatggagatggacaaagctcagagaaagcccctgaggtttctcacatcaaaaagaaagaggtgtccaaaacggtggtcagcaagacttacgagagaggaggggacgtgagccacctcagccacaggtacgagagggatgctggtgtgcttgaacctgggcagccagagaatgacattgacagaatcctccacagccacggctccccaacgcggaggagaaaatgtgccaacctggtgtctgagctaaccaagggctggagagtgatggagcaggaggagcccacatggaggagtgacagcgtagacacagaggacagcggctatggaggagaggctgaggagaggcccgagcaggatggagtgcaggtggctgtggtcaggatcaagcgccccttgccctcccaggtaaacagatttacagagaaactcaactgcaaagcccaacagaaatatagcccagtgggcaacttgaaagggagatggcagcagtgggctgatgaacacatacaatcccagaagctcaatcctttcagtgaagagtttgattacgagctggccatgtccacccgcctacacaaaggagatgagggctatggccgccccaaagaaggaaccaaaactgctgaaagggccaagcgtgctgaggagcacatctacagggaaatgatggacatgtgcttcattatctgcacaatggctcgccacagacgagatggcaagatccaggttacttttggagatctctttgacagatacgttcgtatttcagataaagtagtgggcattctcatgcgtgccaggaaacatggactggtagactttgaaggagagatgctatggcaaggccgagatgaccatgttgtgattacgctactcaagtgaaccttcaaaaacaaaagccaaatttggcccactatggtcttaatgctaaatgctcatgtaggtaaaatgaaaatgcaaactctctgtaataagttagtaaatattaaacaatttttacat

SEQ ID NO: 2 - protein, 381 aa, Homo sapiens
MAPGEKESGEGPAKSALRKIRTATLVISLARGWQQWANENSIRQAQEPTGWLPGGTQDSPQAPKPITPPTSHQKAQSAPKSPPRLPEGHGDGQSSEKAPEVSHIKKKEVSKTVVSKTYERGGDVSHLSHRYERDAGVLEPGQPENDIDRILHSHGSPTRRRKCANLVSELTKGWRVMEQEEPTWRSDSVDTEDSGYGGEAEERPEQDGVQVAVVRIKRPLPSQVNRFTEKLNCKAQQKYSPVGNLKGRWQQWADEHIQSQKLNPFSEEFDYELAMSTRLHKGDEGYGRPKEGTKTAERAKRAEEHIYREMMDMCFIICTMARHRRDGKIQVTFGDLFDRYVRISDKVVGILMRARKHGLVDFEGEMLWQGRDDHVVITLLK